(19) United States
(12) Patent Application Publication
(10) Pub. No.: US 2002/0165610 A1
(43) Pub. Date: Nov. 7, 2002

(54) Ophthalmic lens injectors

(76) Inventor: Terence Arnold Waldock, Meppershall, Bedfordshire (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/030,861

(22) PCT Filed: May 14, 2001
(86) PCT No.: PCT/GB01/02101

(30) Foreign Application Priority Data
May 13, 2000 (GB) 0011507.1

(51) Int. Cl. A61F 2/16

(52) U.S. Cl. 623/6.12

(57) ABSTRACT

An instrument for the insertion of an intraocular lens into an eye comprises a body portion, e.g. a barrel, a nose portion having a lumen through which the lens passes, and a push rod connected to a plunger. Within the nose portion is an insert (30) which defines a passageway (40) therethrough. The passageway has a configuration which includes a smoothly continuous undulating surface (42, 44) upon which a lens rests. A cutout (52) accommodates the trailing haptic. For acrylic lenses, the undulating surface is preferably provided by a recess in the bottom of the passageway, the recess reducing in width and depth to define an upwardly sloping surface on which the lens is deposited.

### FIELD OF THE INVENTION

This invention relates to improvements in ophthalmic lens injectors, and is particularly concerned with lens injectors of the type described in our application WO99/33411.

The lens injectors described in WO99/33411 comprise a body portion, a nose portion through which runs a passage or lumen for the lens to pass to a dispensing tip, and a plunger. The nose portion is pivotally connected to the body portion so that the barrel can be broken open for the placement of the lens into the nose portion. In the aforesaid publication the lens is preferably placed on two spaced parallel nose pins to facilitate its folding. In some of the described embodiments in the aforesaid publication there is provided also a cross pin which straddles the nose pins and under which the intraocular lens is arranged to pass. The purpose of this cross pin is to prevent lifting and tilting of the lens, so that when the plunger pushes it forwards it travels smoothly forwards and is folded properly.

### SUMMARY OF THE INVENTION

It is an object of the present invention to provide improved designs of nose assembly for ophthalmic injectors for injecting intraocular lenses. In the present invention the pins described above are replaced by a specially shaped member within the nose assembly. This member is specially shaped so that it provides an internal surface configuration to assist in guiding the lens into the lumen through which the lens has to pass.

Intraocular lenses may be of silicone or acrylic material. The nose member of the present invention is appropriate for use with both types of lens, although the internal surface configuration may differ, depending on the characteristics of the lens to be inserted. Silicone lenses have haptics which are relatively thin and which are fixed to the lens body. Acrylic lenses have the lens body and haptics made in one piece, with thicker haptics. Also, acrylic material marks more easily than silicone.

In accordance with the present invention there is provided an instrument for the insertion of an intraocular lens into an eye, which comprises a body portion, a nose portion forwardly of the body portion and having a lumen through which the lens is arranged to pass, and push rod means moveable through the body portion and the nose portion to push an inserted lens forwards, wherein there is provided in the nose portion means defining a passageway therethrough, the passageway having a configuration which includes a smoothly continuous undulating surface upon which a lens to be inserted is arranged to rest.

Preferably, the means defining the passageway is an insert having an external configuration which makes it non-rotatable within the nose assembly. In a preferred embodiment the insert has a forwardly projecting portion shaped to match the internal configuration of the lumen through the nose assembly and providing guide means for the forward guidance of the lens.

In one embodiment, particularly suitable for silicone lenses, the undulating surface comprises two upstanding arcuate ribs at the bottom of the passageway. Preferably, in order to ensure that the trailing haptic remains free, there is provided a cutout to accommodate the trailing haptic at one side of the bottom of the passageway.

In another embodiment, particularly suitable for acrylic lenses, the undulating surface is provided by a recess in the bottom of the passageway. This enables a lower entry of the lens into the chamber defined by the passageway and keeps the lens away from the upper surface of the passageway.

### BRIEF DESCRIPTION OF THE DRAWINGS

In order that the invention may be more fully understood, a number of presently preferred embodiments of lens injector in accordance with the invention will now be described in more detail by way of example and with reference to the accompanying drawings, in which:

FIG. 1 is a side view of a lens injector in accordance with the invention incorporating a nose assembly of the present invention;

FIG. 2 is a top plan view of the lens injector of FIG. 1, with the plunger retracted;

FIG. 3 is a view from the other side of the lens injector of FIG. 1, with the plunger retracted;

FIG. 4 is a side view of the nose of the lens injector;

FIG. 5 is an end view of the nose of FIG. 4, viewed from the right-hand end of FIG. 4;

FIG. 6 is a view from the other side of the nose of FIG. 4;

FIG. 7 is a top plan view of the nose of FIG. 4;

FIG. 8 is an end view of the nose as shown in FIG. 7, viewed from the right-hand end of FIG. 7;

FIG. 9 is a side view of a first embodiment of nose insert for insertion into the nose of FIGS. 4 to 8;

FIG. 10 is an end view of the nose insert of FIG. 9, viewed from the right-hand end of FIG. 9;

FIG. 11 is a side view of a second embodiment of nose insert;

FIG. 12 is an end view of the nose insert of FIG. 11, viewed from the right-hand end of FIG. 11;

FIG. 13 is a side view of a third embodiment of nose insert;

FIG. 14 is an end view of the nose insert of FIG. 13, viewed from the right-hand end of FIG. 13;

FIG. 15 is a plan view of the nose insert of FIGS. 13 and 14;

FIG. 16 is the side view of FIG. 13, showing the loading forceps in position;

FIG. 17 is a side view of the centre rod used with the nose insert of FIGS. 9 and 10;

FIG. 18 is a side view of the centre rod used with the nose insert of FIGS. 11 and 12; and,

FIG. 19 is a side view of the centre rod used with the nose insert of FIGS. 13 to 15.

### DESCRIPTION OF THE PREFERRED EMBODIMENTS

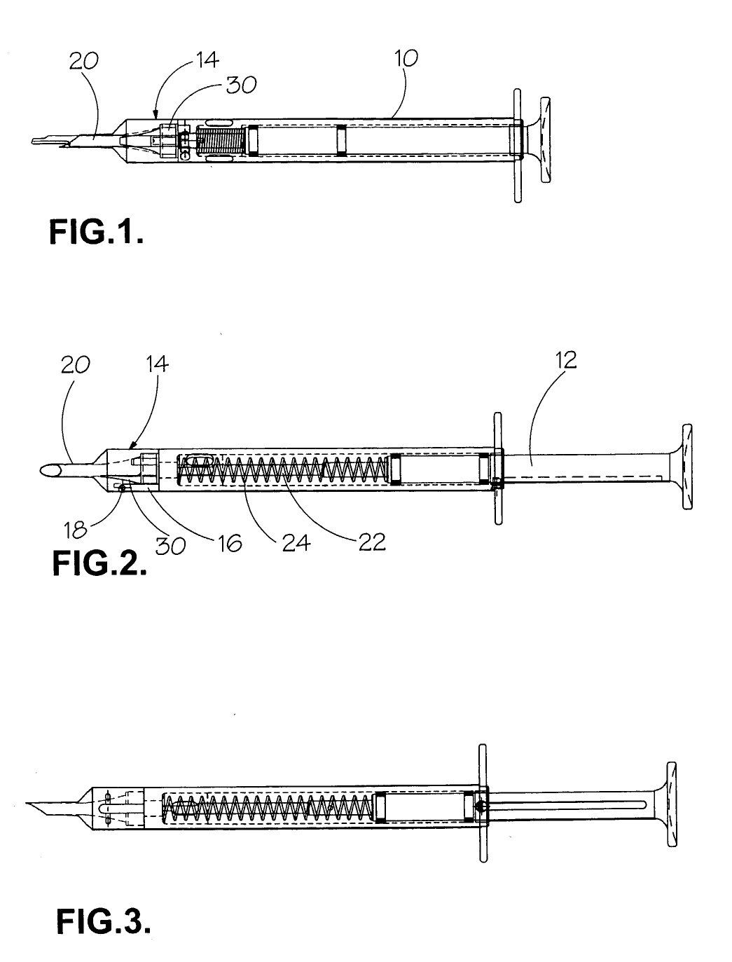

Referring first to FIGS. 1 to 3, the intraocular lens injector shown there is of the general type described and shown in our patent application PCT/GB98/03917 (WO99/33411) which has a nose portion which can be “broken open” in like manner to a shotgun barrel. However, it is to be understood that the present invention is applicable to other types of intraocular lens injectors, and that the embodiments of nose assembly are described in relation to this particular type of lens injector by way of example only.

The lens injector as shown in the drawings essentially comprises a body portion 10, a plunger 12 and a nose indicated generally at 14. The nose 14 can be “broken open” in like manner to a shotgun barrel. In the closed position as shown in the drawings the nose 14 is coaxial with the main body 10 and the plunger 12. The body portion 10 has a finger 16 projecting from the front end of the body at the bottom of the body, and a pivot pin 18 extends through the nose and the finger to provide the pivotal mounting. The nose is pivotable through 90° from the open position to the closed position and vice versa.

The nose 14 will be described in more detail hereinafter. Suffice it to say here that there is a passage completely through the nose which changes in cross-section and configuration from one end of the nose to the other. At the distal end the nose has a tip 20 through which the lens is ejected. As shown in FIG. 1, when the plunger 12 is fully depressed, a centre rod 22, which is secured to the plunger, passes out through the tip of the nose. The plunger 12 is slidable within the body portion 10 which is in the form of a cylindrical barrel having a bore therethrough. A spring 24 provides a force against which the plunger is depressed and which urges the plunger into its retracted position as shown in FIGS. 2 and 3. For further details of the structure of the lens injector, reference should be made to the aforesaid application WO99/33411.

The nose assembly 14 includes, as stated, a tip portion 20 which is shaped at its outer end to form a dispensing aperture of appropriate shape and orientation. Through the nose 14 there extends an internal passageway 26 along which the lens passes under the action of the centre rod or push rod 22, embodiments of which are shown most clearly in FIGS. 17 to 19.

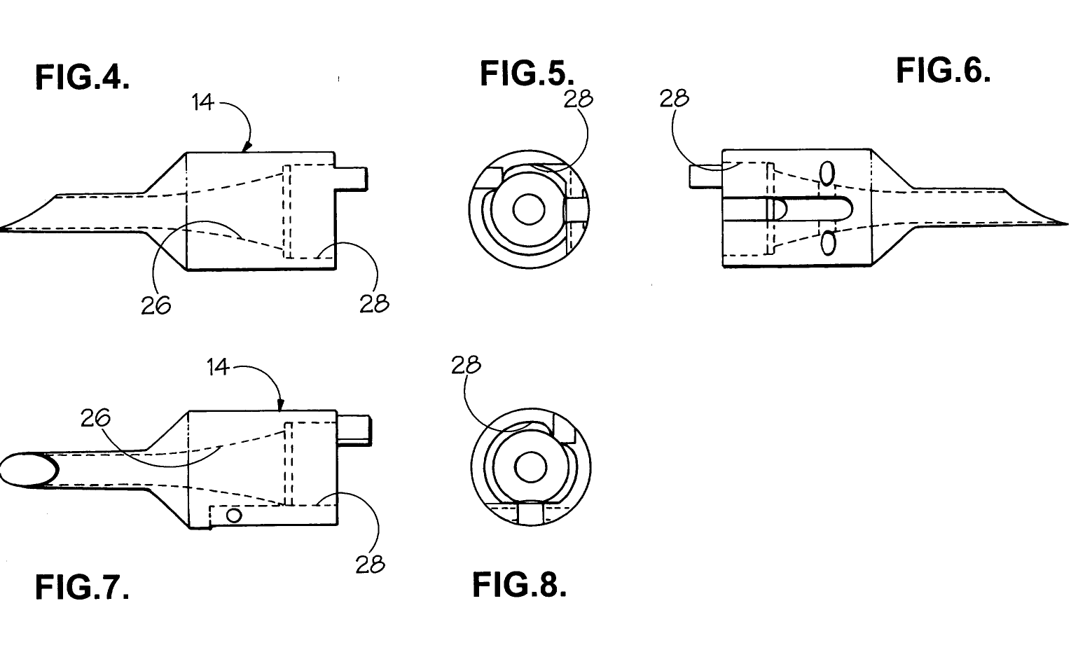

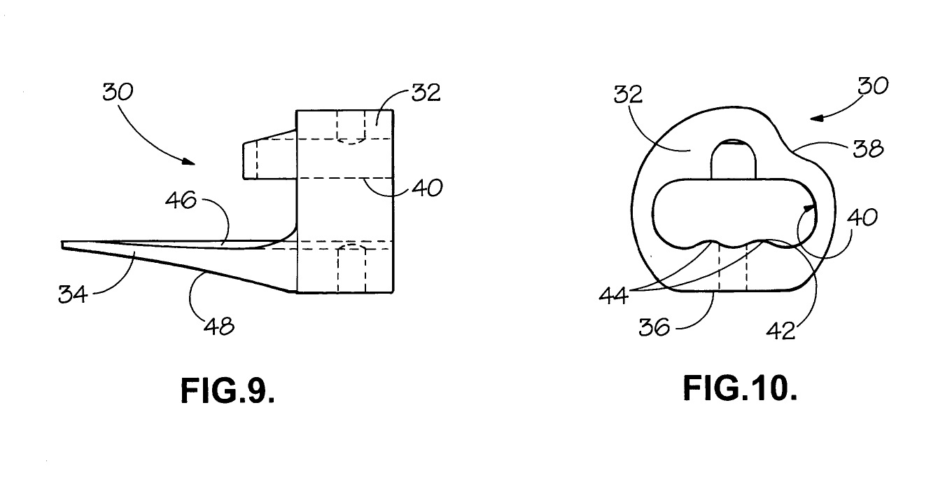

The internal shape and configuration of the nose 14 can be seen from FIGS. 4 to 8. The rearward end of the nose 14 is recessed, as indicated at 28, to receive a nose insert 30 which is shown most clearly in FIGS. 9 and 10, but which can also be seen, in position, in FIGS. 1 and 2. The cross-sectional configuration of the recess 28 in the nose 14 matches the external configuration of the insert 30 shown in FIGS. 9 and 10. Thus, the insert 30 as shown in FIG. 10 will fit directly into the recess 28 as presented in FIG. 8. The inside surface of the taper and of the ongoing bore of the passageway 26 through the nose 14 must have a good machined finish with a smooth transition between the taper and the subsequent bore.

The insert 30 is shown most clearly in FIGS. 9 and 10. It is shaped and configured to permit the dispensing of the lens reliably and effectively through the nose without the use of pins to support and guide the lens. The nose insert shown in FIGS. 9 and 10 is particularly suited for the insertion of silicone lenses. The insert 30 is a one-piece element, for example of titanium, shaped to fit within the nose 14. It comprises a shoulder portion 32 and a forwardly projecting arm 34. The shoulder portion 32 has one face 36 which is flat and its otherwise circular periphery is indented at 38 for engagement by a pin to prevent rotation of the insert 30 within the nose 14. The shoulder 32 has a bore 40 therethrough. This is generally rectangular in cross-section as shown most clearly in FIG. 10, but with the bottom surface 42 having a smoothly continuous undulating shape defining two upstanding arcuate ribs 44. The lens to be inserted is placed in this bore 40 on the ribs 44 using forceps. The forwardly projecting arm 34 of the insert 30 is provided with a guide channel 46 which forms a continuation of the guide surface 42, for the onward passage of the lens through the nose and into the tip. The underside 48 of the arm 34 as shown in FIG. 9 is concavely curved to match the configuration of the tapered passageway 26 within the nose 14.

Shaping the nose insert recess 40 in this way, and extending this into a tapered bore 26 through the nose, causes the lens which is inserted to be folded as it is pushed forward by the push rod 22 into the passage through the tip. The folding of the lens is effected by the shape of the encircling passageway, namely by the undulations 44 and the taper of the passageway.

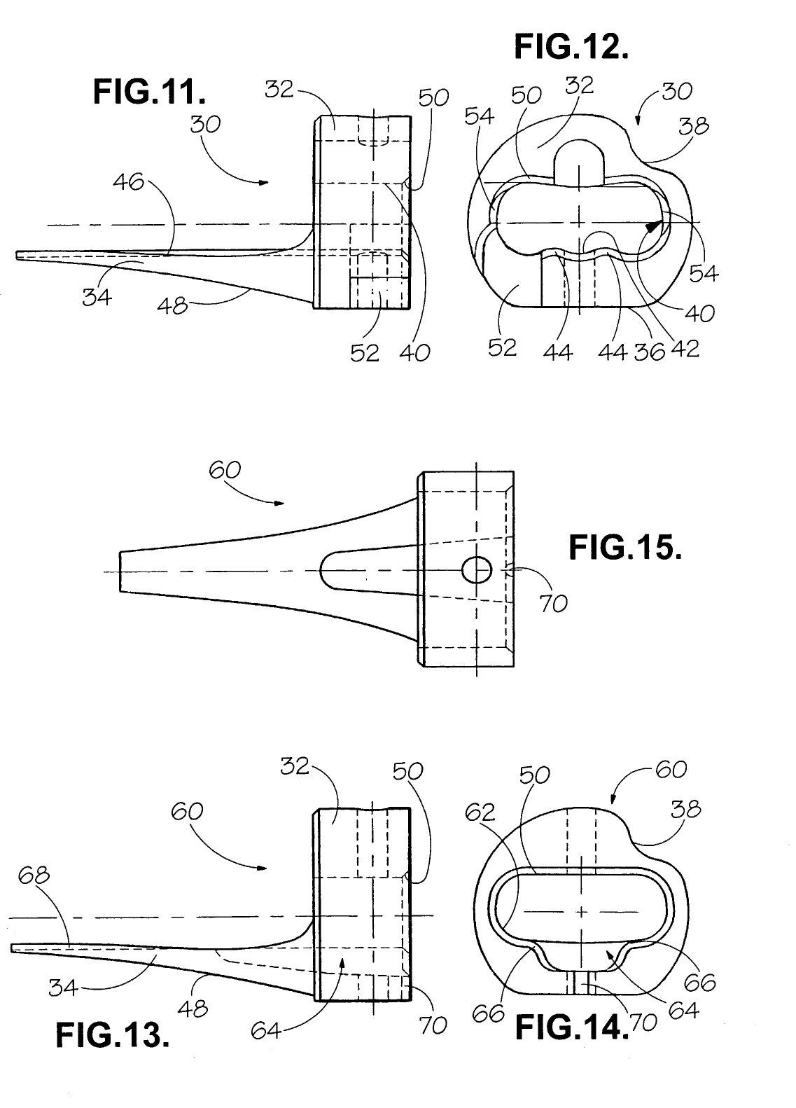

A second embodiment of nose insert is shown in FIGS. 11 and 12. The same or corresponding features shown in FIGS. 9 and 10 are indicated by the respective same reference numerals. In this embodiment, the internal shape of the passageway 40 is generally the same as in FIGS. 9 and 10, although the upper surface of the passageway is here slightly convex downwards. Also, the opening to the passageway 40 is here shown as chamfered at 50. The main difference in the embodiment shown in FIGS. 11 and 12 is that a cutout 52 is provided in the input end face of the insert, from the exterior of the insert to one bottom corner of the passageway 40, adjacent to one of the arcuate ribs 44. This cutout 52 is to accommodate the trailing haptic, so that there is a reduced danger of this becoming trapped or otherwise being caught up upon the insertion of the lens with the forceps.

The nose insert 30 of FIGS. 11 and 12 is particularly suited for use with silicone lenses. In order to enable the nose insert of FIGS. 11 and 12 to accept a 6 mm silicone lens, two small recesses 54 are provided in the passageway 40, one on each lateral side of the passageway. These provide extra width for the 6 mm lens to be deposited in the nose insert.

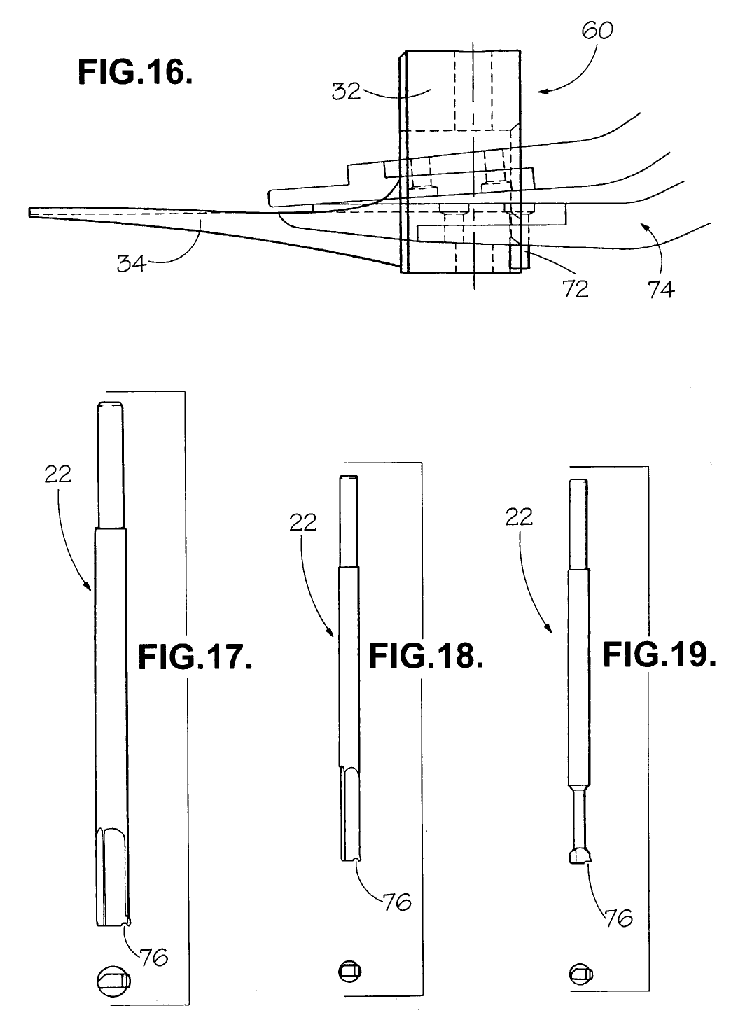

FIGS. 13 to 15 show a third embodiment of nose insert, here indicated generally at 60. If one is dealing with acrylic lenses, where the lens body is integral with the haptics, the haptics are thicker than in the case of a silicone lens. Also the acrylic material marks more easily than silicone. It is therefore desirable to be able to keep the lens low on insertion into the passageway of the nose insert and in particular to keep the lens away from the upper surface of the passageway. A nose insert which enables this to be achieved is shown in FIGS. 13 to 15. Here the passageway through the shoulder 32 of the nose insert is indicated at 62 and again has a chamfered surround 50. However, as compared with the second embodiment, the configuration of the bottom surface of the passageway 62 is different. It is here recessed as indicated at 64. As shown most clearly in FIGS. 13 and 15, the recess 64 extends through the shoulder portion 32 and into the projecting arm 34 of the insert, becoming shallower and tapering in width. The undulating smoothly continuous bottom surface of the passageway 62 is therefore here formed by lateral shoulders 66 and the intermediate recess 64. This configuration enables the lens to be placed with the forceps low in the chamber and in a way which causes it to be folded as it is pushed forward. In this embodiment, only the forward end of the arm 34 is provided with a guide channel 68. Below the recess 64, there is provided a notch 70 in the face of the insert. This notch 70 is arranged to receive a pin 72 which projects from the underside of the forceps used to insert the lens. FIG. 16 shows the insert 60 with a pair of forceps 74 in position within the insert, positioned to deposit the lens within the chamber. Using the forceps 74 in this way, the lens is deposited on an “up” slope, namely the bottom of the recess 64 which slopes upwards from the front face of the nose insert towards the nose tip. This gives a more reliable and effective deposition of the lens, particularly with an acrylic lens.

FIGS. 17 to 19 show three embodiments of centre rod 22 which are used respectively for the pushing of the lens forwards and out of the nose insert as shown in each of the three embodiments described above. In each case the centre rod is indicated at 22. The rear end of each centre rod is connected to the plunger 12. The shape and configuration of the front end of the push rod 22 is in each case designed to enable it to engage and hold the lens in its forward movement through the lumen. As shown, the front end is shaped to define an engaging recess 76 which “picks up” the lens when placed within the chamber in the insert. The recess 76 is preferably trapezoidal in shape in the side view as shown. As shown in FIG. 1, when the plunger is fully depressed, the push rod 22 extends fully out through the tip 20 to enable the lens to be inserted into an incision in the eye.

1. An instrument for the insertion of an intraocular lens into an eye, which comprises a body portion, a nose portion forwardly of the body portion and having a lumen through which the lens is arranged to pass, and push rod means moveable through the body portion and the nose portion to push an inserted lens forwards, wherein there is provided in the nose portion means defining a passageway therethrough, the passageway having a configuration which includes a smoothly continuous undulating surface upon which a lens to be inserted is arranged to rest.

2. An instrument according to claim 1, in which the means defining the passageway is an insert having an external configuration which makes it non-rotatable with the nose portion.

3. An instrument according to claim 2, in which the insert has a forwardly projecting portion shaped to match the internal configuration of the lumen through the nose portion and providing guide means for the forward guidance of the lens.

4. An instrument according to any preceding claim, in which the undulating surface comprises two upstanding arcuate ribs at the bottom of the passageway.

5. An instrument according to claim 4, in which the ribs are equispaced either side of the centre of the passageway, with a depression between them and depressions at the outside of each rib.

6. An instrument according to claim 4 or 5, in which a cutout is provided in the means defining the passageway to accommodate the trailing haptic of a lens.

7. An instrument according to claim 6, in which the cutout is an extension of the passageway at one side of the bottom of the passageway adjacent to one of said ribs.

8. An instrument according to claim 1, 2 or 3, in which the undulating surface is provided by a recess in the bottom of the passageway, bounded by a shoulder on each side.

9. An instrument according to claim 8, in which the recess reduces in depth and width in the direction away from the push rod means, to define an upwardly sloping surface on which the lens is to be deposited.

10. An instrument according to claim 2 or 3, in which the insert is provided with means to locate a pair of forceps when a lens is deposited in the passageway.

11. An instrument according to any preceding claim, in which the nose portion is pivotable through 90° relative to the body portion.

12. An instrument according to any preceding claim, in which the lateral sides of the passageway are recessed to accommodate a lens of 6, mm diameter.
